SEQUENCE LISTING (4 sequences)

SEQ ID NO: 1 - protein, 43 aa, Homo sapiens
DAEFRHDSGYEVHHQKLVFFAEDVGSNKGAIIGLMVGGVVIAT

SEQ ID NO: 2 - protein, 40 aa, Homo sapiens
DAEFRHDSGYEVHHQKLVFFAEDVGSNKGAIIGLMVGGVV

SEQ ID NO: 3 - protein, 42 aa, Homo sapiens [sequence omitted]

SEQ ID NO: 4 - protein, 5 aa, Homo sapiens
KLVFF

(19) United States
(12) Patent Application Publication
(10) Pub. No.: US 2006/0069010 A1
(43) Pub. Date: Mar. 30, 2006

(54) Detoxification depot for Alzheimer's disease

(75) Inventors: Stanley Stein, East Brunswick, NJ (US); Pazhani Sundaram, Cheshire, CT (US); Chinnaswamy Kasinathan, Holmdel, NJ (US)

Correspondence Address: MCCORMICK, PAULDING & HUBER LLP, CITY PLACE II, 185 ASYLUM STREET, HARTFORD, CT, 06103, US

(73) Assignee: Senicure LLC, Mount Olive, NJ (US)

(21) Appl. No.: 10/966,919

(22) Filed: Oct. 15, 2004

Related U.S. Application Data
(60) Provisional application No. 60/511,674, filed on Oct. 17, 2003.

(51) Int. Cl. A61K 38/17; A61K 9/14

(52) U.S. Cl. 514/2; 424/486

(57) ABSTRACT

The invention is directed to a device that is placed inside an Alzheimer's disease (AD) patient for the purpose of extracting and accumulating neurotoxic beta-amyloid peptides (nt-bAP) from body fluids. AD is the consequence of a process in which nt-bAP aggregates to form fibrils and plaques which can cause nerve damage. Since nt-bAP can cross the blood-brain barrier (BBB), the concentration in the central nervous system and in the periphery are in equilibrium. By sequestering nt-bAP, our device will act as a “sink.” It should draw nt-bAP across the BBB, reducing the concentration of soluble nt-bAP in the brain, thereby halting or slowing plaque deposition in the brain. Since plaques and possibly soluble, aggregated nt-bAP are the cause of nerve damage in AD, this process should be therapeutically effective. The device can be a depot containing a fragment of nt-bAP which intrinsically retains the ability to bind but not to be toxic.

### CROSS-REFERENCE TO RELATED APPLICATIONS

This application is entitled to the benefit of and incorporates by reference essential subject matter disclosed in U.S. Provisional Patent Application No. 60/511,674 filed on Oct. 17, 2003.

### FIELD OF THE INVENTION

This invention concerns a device that is implanted, such as under the skin, for treating patients with Alzheimer's disease. The device may function as a long-acting detoxification depot, based on its ability to bind and retain the neurotoxic amyloid peptides in the brain. The depot will act as a “sink,” causing soluble neurotoxic amyloid peptides to cross the blood-brain barrier, thereby halting or reversing these plaques in the brain.

### BACKGROUND

The hallmark of Alzheimer's disease (AD) is the presence in the brain of senile plaques, which are composed of a central deposition of β-amyloid peptide. Genetic, neuropathological and biochemical evidence has shown that these deposits of β-amyloid peptide play an important role in the pathogenesis of AD. β-Amyloid (Aβ) peptide refers to a 39-43 amino acid peptide derived from the amyloid precursor protein (APP) by proteolytic processing (FIG. 1). Both Aβ¹⁻⁴⁰ and Aβ¹⁻⁴² are components of the deposits of amyloid fibrils found in brain tissue of AD patients. The aggregation of monomeric Aβ peptides into toxic fibrils and plaques has a rate-limiting nucleation phase followed by rapid extension. Aβ¹⁻⁴² is believed to play a more important role in the early nucleation stage, thus being more “amyloidogenic” than Aβ¹⁻⁴⁰.

The earliest studies of the aggregation process identified the critical region of Aβ involved in amyloid fibril formation by altering the hydrophobic amino acids in Aβ by substituting more hydrophilic amino acids and testing the effects of these changes. Their results showed that the hydrophobic core at residues 17-20 of Aβ, LVFF, is crucial for the formation of the β-sheet structure and the amyloid properties of Aβ. The Aβ¹⁻⁴⁰ analogues, in which the amino acids in 17-20 are replaced by more hydrophilic amino acids, are still able to bind to full length Aβ¹⁻⁴⁰. Furthermore, they were reported to inhibit fibril formation in vitro and, therefore, these analogues were suggested as therapeutic reagents for AD. Similarly, synthesized numerous peptide fragment of the Aβ¹⁻⁴⁰ molecule and found that the shortest peptide still displaying consistently high Aβ binding capacity had the sequence KLVFF (corresponding to Aβ¹⁶⁻²⁰). This peptide was studied by microscopy and was found to be able to interfere with fibril formation in vitro. Having shown that the short peptide KLVFF can bind to Aβ and disrupt ordered fibril formation, showed that peptide KLVFF binds to the homologous sequence in Aβ, i.e. Aβ¹⁶⁻²⁰. Also, molecular modeling suggested that association of the two homologous sequences leads to the formation of an atypical anti-parallel β-sheet structure stabilized primarily by interaction between the Lys, Leu and Phe residues. The self-recognition property of the peptide, KLVFF has recently been confirmed.

Based on these results, it was developed that employed an approach to the design of inhibitors of Aβ toxicity a recognition element, which interacts specifically with Aβ, is combined with a disrupting element, which alters Aβ aggregation pathways. They synthesized a peptide composed of residues 15-25 of Aβ, designated as the recognition element, linked to an oligolysine β-sheet disrupting element. This inhibitor does not alter the apparent secondary structure of Aβ nor prevent its aggregation; rather, it causes changes in aggregation kinetics and higher order structural characteristics of the aggregate. In addition to its influence on the physical properties of Aβ aggregates, the inhibitor completely blocks Aβ toxicity to neuron-like PC-12 cells. These results suggest that formation of disordered aggregates rather than complete blockade of amyloid fibril formation might be sufficient for abrogation of toxicity.

Many peptide fragments, homologous to the β-amyloid peptide, have been synthesized and tested, and they can block the orderly aggregation of the β-amyloid peptide. Small peptides were designed to interfere with the development of β-sheet structures β-sheet breaker, a pentapeptide with partial homology to the β-amyloid peptide, was shown to be capable of preventing β-amyloid fibril formation and disassembling preformed fibrils in vitro when a 20-fold excess of inhibitor peptide was used. However, specific binding to plaques was not shown. More recently, a peptidase-resistant congener based on the KLVFF motif, having N-methyl amino acids at alternate positions, was shown to prevent ordered fibril formation. Although interesting, the ability of these β-sheet breakers to oppose the accumulation of toxic plaques has been demonstrated only in model in vitro systems. To be useful therapeutically, these inhibitory compounds must be able to cross the blood-brain barrier (BBB). Furthermore, there must be specificity in the ability of the proposed inhibitory compounds to recognize aggregates of β-amyloid peptide, rather than bonding and disrupting β-sheet structures in unrelated proteins.

Two recent publications have brought attention to another potential approach for preventing or at least minimizing the accumulation of plaques. In both articles, the authors suggest that Aβ peptides can cross the blood-brain barrier (BBB) and therefore will establish an equilibrium of Aβ in the central nervous system (CNS) and the periphery. In one report monoclonal antibodies to Aβ were injected peripherally at a high dose (0.5 mg) into AD model mice. Plasma levels of Aβ were measured (including both free and antibody-bound Aβ). Prior to administering the antibody, Aβ levels in blood were quite low (ca. 0.25 ng/ml) irrespective of the amyloid burden in the brain. In contrast, 24 hours after administering the antibody, plasma level increased between 10 and 50-fold, and this increase correlated with the amount of amyloid plaque in the brain. Supposedly, the relatively large amount of Aβ came from the brain, implying that Aβ can cross the BBB with the monoclonal antibody acting as a “peripheral sink.” With a plasma volume of only several milliliters, the amount of Aβ drawn out of the brain was on the order of tens of nanograms. The second article corroborated these findings [15]. Instead of using a monoclonal antibody, they used a protein, gelsolin, and a lipid, GM1, both of which have a propensity to bind Aβ. In their studies with AD model mice, they demonstrated the levels of Aβ in brain could be reduced in half (on the order of 500 ng/g tissue) even after 1 day of treatment.

### SUMMARY OF THE INVENTION

The Invention is a device that can be implanted into an AD patient and will absorb and concentrate nt-bAP in a harmless form. In a preferred embodiment, the device comprises a matrix of cross-linked poly(ethylene glycol), which can be injected as a liquid but will form a hydrogel. This depot is in good contact with body fluids while otherwise being essentially inert (1, Qiu et al). The depot also includes a capture reagent for nt-bAP, such as a monoclonal antibody or a KLVFF-related peptide as described (2, Zhang et al). Whereas Qiu et al. concerned a device for delivery of therapeutic agents in a long-acting manner, the present Invention uses the same gel in a unique manner, to capture and sequester toxic substances. Zhang et al. teaches that specific binding interactions with nt-bAP can be obtained using just a pentapeptide, reasoning that the specificity for a particular target increases as the size of the binding element decreases. Zhang et al. also teaches that the avidity of binding can be increased by linking together multiple copies of the binding element. Zhang et al. also teaches that the retro-inverso peptide, ffvlk, can comprise this binding element, imparting 2 favorable properties: stability against degradation and making aggregates of the binding element with nt-bAP less toxic than nt-bAP itself, according to the thioflavin assay. Thus, the Invention is unique, being derived from two otherwise unrelated technologies (Qie et al. and Zhang et al.).

Another consideration in this invention is a means to remove the depot after it is no longer functional. The gel may simply be surgically removed or it may be constructed to autodegrade. As a precaution, the depot may also be loaded with a protease or peptidase that will degrade captured beta-amyloid peptide into nontoxic fragments. Alternatively, fragments of the depot or physically trapped polymer or monoclonal antibody may be designed to help eliminate beta-amyloid peptide from the body via the liver. An attribute of the retro-inverso peptides described by Zhang et al. is that the aggregates formed with nt-bAP might not be neurotoxic, according to the thioflavin fluorescence test. Dimers and higher order repeats of the binding peptides might require only one attachment site to the matrix or may just be physically trapped in the depot, which might be helpful for their elimination from the body.

Thus, the Invention comprises the following components:
a biocompatible matrix such as made by cross-linking poly(ethylene glycol) polymers to form a hydrogel through which water and other substances can diffuse in and out;
a capture reagent for nt-bAP, which can be a monoclonal antibody or a fragment or analog of nt-bAP (e.g. retro-inverso peptides such as phe-phe-val-leu-lys) that is linked to the matrix;
which together could actually trap nt-bAP.

### BRIEF DESCRIPTION OF THE DRAWINGS

FIG. 1 illustrates partial sequence of APP770. The β-amyloid peptide, .Aβ¹⁻⁴³, is shown in bold italics; Aβ¹⁻⁴⁰ would have IAT truncated from the C-terminus. KLVFF is underlined.

FIG. 1a graphically illustrates binding of biotinylated Aβ¹⁻⁴² and biotinylated Aβ¹⁻⁴⁰ peptide by RI peptide. 96-well plate was coated with the capture peptide (1 μg/well of retro-inverso [RI], scrambled [SCR] or an irrelevant control peptide), blocked with gelatin and probed with 1 μg/ml of biotinylated Aβ peptide for 2 hours and streptavidin peroxidase for 1 hour. Experiments were repeated and were calculated as Mean±SE (N=2) and expressed as pmols Aβ¹⁻⁴²/ml of binding solution. Graded concentrations of biotinylated Aβ¹⁻⁴² peptide was used as a calibration standard.

FIG. 2a graphically illustrates binding of biotinylated Aβ¹⁻⁴² peptide to detox gel. Binding experiment performed with the detox gel (RI gel) and control gels as denoted. Binding assay was performed as described in the methods section. Pre-swelled individual gels were incubated in the binding solution containing phosphate buffer (10 mM, pH 7), biotinylated Aβ¹⁻⁴² peptide (1.7 μg/ml) at 37° C. Samples were harvested at 0, 15, 30, 60, 120 and 180 minutes. Then the gels were washed and incubated in buffer containing no biotinylated Aβ¹⁻⁴² peptide for up to 4 days at 37° C. Samples were collected at the end of 1 and 4 days to assess the release of the biotinylated Aβ¹⁻⁴² peptide back into the medium. Harvested samples were plated on a 96 well plate and ELISA performed to quantitate the biotinylated Aβ¹⁻⁴² peptide. Experiments were repeated and were calculated as Mean±SE (N=3) and expressed as pmols Aβ¹⁻⁴²/ml of binding solution. Graded concentrations of biotinylated Aβ¹⁻⁴² peptide was used as a calibration standard. After completing and reporting this study, we found a recent article (19) showing that a scrambled peptide can be an active binder too.

FIG. 2b graphically illustrates binding of biotinylated Aβ¹⁻⁴² peptide to detox gel. Binding experiment with the detox gel (RI gel) or control gel was performed as described in the methods section. Pre-swelled individual gels were incubated in the binding solution containing phosphate buffer (10 mM, pH 7), biotinylated Aβ¹⁻⁴² peptide (1.7 μg/ml) at 37° C. Samples were harvested at 0, 30, 45, 90 and 120 minutes. Harvested samples were plated on a 96 well plate and ELISA performed to quantitate the biotinylated Aβ¹⁻⁴² peptide. Experiments were repeated and were calculated as Mean±SE (N=3) and expressed as pmols Aβ¹⁻⁴²/ml of binding solution. Graded concentrations of biotinylated Aβ¹⁻⁴² peptide was used as a calibration standard.

FIG. 3 graphically illustrates binding of biotinylated Aβ¹⁻⁴⁰ peptide to detox gels. Binding experiment with the detox gel (RI gel) and control gel was performed as described in the methods section. Pre-swelled individual gels were incubated in a pre-coated 48-well plate with the binding solution containing phosphate buffer (10 mM, pH 7), biotinylated Aβ¹⁻⁴⁰ peptide (1.7 μg/ml) at 37° C. Samples were harvested at 0, 15, 30, 45, 60, 90 and 120 minutes. Then the gels were washed and incubated in buffer containing no biotinylated Aβ¹⁻⁴⁰ peptide for 18 hours at 37° C. to assess the release. Harvested samples were plated on a 96 well plate and ELISA performed to quantitate the biotinylated Aβ¹⁻⁴⁰ peptide. Experiments were repeated and were calculated as Mean±SE (N=3) and expressed as pmols Aβ¹⁻⁴⁰/ml of binding solution. Graded concentrations of biotinylated Aβ¹⁻⁴⁰ peptide was used as a calibration standard.

FIG. 4 graphically illustrates binding of biotinylated Aβ¹⁻⁴² peptide to different formulation of detox gels. Individual detox gels were made to contain 2%, 4% or 5% PEG and a fixed RI peptide concentration. Binding experiment with the detox gels was performed as described in the methods section. Pre-swelled individual gels were incubated in the binding solution containing phosphate buffer (10 mM, pH 7), biotinylated Aβ¹⁻⁴² peptide (1.7 μg/ml) at 37° C. Samples were harvested at 0, 15, 30, 45, 60, 90 and 120 minutes. Harvested samples were plated on a 96 well plate and ELISA performed to quantitate the biotinylated Aβ¹⁻⁴² peptide. Experiments were repeated and were calculated as Mean±SE (N=3) and expressed as pmols Aβ¹⁻⁴²/ml of binding solution. Graded concentrations of biotinylated Aβ¹⁻⁴² peptide was used as a calibration standard.

### DETAILED DESCRIPTION OF THE PREFERRED EMBODIMENTS

We propose to base our new therapeutic strategy on this new “brain to plasma efflux” approach. We suggest that the KLVFF-related peptides presented in Zhang et al. [2] and in PCT/US02/26889 would be superior to monoclonal antibodies, gelsolin or GM1 in this therapy. The KLVFF-related peptides could be monomers, dimmer, trimers or higher oligomers linked to one another in a linear or branched form, such as, but not limited to Table 1:

TABLE 1
KLVFF-related peptides

| Structure of conjugate | Copies of peptide |
|---|---|
| Lys-Leu-Val-Phe-Phe-Cys | 1 (native) |
| phe-phe-val-leu-lys-cys | 1 (retro-inverso) |
| [phe-phe-val-leu-lys-βAla]₂-lys-cys (branched) | 2 (retro-inverso) |
| [phe-phe-val-leu-lys-βAla]₄-lys₂-lys-cys (branched) | 4 (retro-inverso) |
| [phe-phe-val-leu-lys-PEG-lys-]₃-cys (linear) | 3 (retro-inverso) |

Lower case is for D-amino acids. βAla is beta-alanine, C-terminus is amidated, uncharged form, N-terminus is free, positive charged form, PEG can be terminated by an amino group at one end and a carboxylate group at the other end. In a preferred embodiment, the cysteine residue is linked via its side chain thiol to the gel matrix.

Many functionalized forms of the relatively inert polymer, poly (ethylene glycol) (PEG) are commercially available, allowing numerous methods for linking other substances to PEG molecules [16-18]. The complementary linker group for a thiol could be a maleimide or vinylsulfone group for a non-reducible thioether bond or another thiol for a reducible disulfide bond. We have been developing hydrogel (defined as being>90% water) composed of PEG as sustained-release drug delivery systems [19]. These hydrogels have been kept as subcutaneous depots in rabbits for up to 6 months without any sign of toxicity [19]. Aqueous solutions of the formulation components can be mixed in a syringe and will form a hydrogel in a precise time period (usually about 1 minute), allowing easy and reliable injection. If necessary, the gel “button” can be removed by making a small incision in the skin. The hydrogel is in good contact with the interstitial fluid. The porosity of the gel can be adjusted; for example, a 4% hydrogel will exclude linear dextran above 300 kDa (unpublished results). With the versatility provided by the modified forms of PEG, it is possible to covalently attach drug molecules using bioreversible bonds, such as ester and disulfide. Similarly, autodegradation of the hydrogel can be designed. Based on these and other favorable properties, we now propose to use the hydrogel as a detoxification depot. The different steps involved in plaque formation and the proposed mechanism of action of “detoxification depot” are as follows:

STEP 1. APP is produced in the brain

STEP 2. APP is degraded into fragments; the two fragments known as Aβ¹⁻⁴² and Aβ¹⁻⁴⁰ are potentially neurotoxic when they form aggregates.

STEP 3. Under normal circumstances, the rate of production of nt-b AP is equal to its rate of removal from the central nervous system. In AD the rate of removal is less than the rate of production and excess nt-b AP forms plaque.

STEP 4. Placement of a detoxification depot in the periphery will augment the rate of removal of nt-b AP from the CNS, thereby halting plaque formation.

We combined the features of the Aβ binding agents with the properties of the subcutaneous hydrogel drug delivery system into a novel therapeutic system. This system will be able to accumulate soluble Aβ, preventing its deposition as plaque in the brain, thereby halting progression of Alzheimer's disease. It is inherently clear, from the body of information described and referenced above, that the present invention can provide a therapeutic product for AD. Anyone or any group of individuals skilled in the art of pharmaceutical practice should be able to prepare and use the present invention for AD therapy. In the following Examples, we describe methods for preparing the detoxification depots. We then demonstrate experimentally, in vitro, the ability of these detoxification gels to capture and retain nt-bAP.

### EXAMPLE 1

Strategies used for preparation of gels. Gels were made in a manner similar to that of Qiu et al. (2003) [1] by using, PEG-NH2, 8-arm and VS-PEG-NHS as polymer and copolymer, respectively. SH-PEG-SH, synthesized as described below, served as the linker. The linker links the conjugated PEG 8-arm together and if it was used at the right concentration, linking of the 8-arm PEGs were achieved. Empty gels were made using these components. For the detox gels, retro-inverso peptide was attached to 2-3 arms of the 8-arm PEG. Then the peptide was reacted with VS-PEG-NHS and the gel was made with the linker, SH-PEG-SH. The vinyl sulfone (VS) group has desirable properties of rapid and selective reaction with thiol (—SH) groups and stability in water, both at neutral pH. The binding element, retro-inverso peptide (RIP), phe-phe-val-leu-lys-Cys was composed of D-amino acids. A ‘Cys’ was placed at the C-terminus of the peptides to utilize its thiol group for linkage. The cysteine thiol group was used for appending the peptide to the gel matrix. The strategy was to place the RI at the end of a long PEG chain, thereby allowing it freedom of motion within the hydrogel, which was greater than 90% water. As a result, the RIP should be able to form the multimeric aggregates needed for high affinity binding of toxic amyloid peptides. Positive and negative control gels were made the same way by replacing the RI peptide with native or scrambled peptides (described below), respectively.

Preparation of gels. Empty gels were made by using, 8-arm PEG-NH2, (MW-10, 000) and VS-PEG-NHS (MW-3, 400, both from Nektar Therapeutics Inc, AL). For 2% gel, approximately 1.5×10 ⁻⁶ moles of PEG was used. VS-PEG-NHS at 1.2-fold molar ratio was added to PEG 8-arm solution very slowly (drop-wise) and mixed by light shaking and left at room temperature for 2 hours for reaction to be complete. This reaction produced PEG-VS₈, which was distributed into 1.5 ml polypropylene tubes. Then for detox gels, 4×10⁻⁷ moles of peptide per gel was added to appropriate tubes and the reaction allowed to go for 6 hours. This way, they were attached to 2-3 arms of the 8-arm PEG. At this point we had PEG-Peptide₃-VS₅. For empty (control) gel, PB (20 mM, pH=8.0) was used. Reaction was allowed to proceed for 2-12 hours. For the last step linker was added. 3.25×10⁻⁷ moles of “disulfide-linker” (HS-PEG _{3,400}-SH) was added to each tube and mixed to form the gels. Gels were stored at 4° C., soaked in PB containing 0.005% sodium azide. Each gel was 100 μl in volume and was made in 1.5 ml polypropylene tube. 2% (PEG) gels were used in most experiments.

### EXAMPLE 2

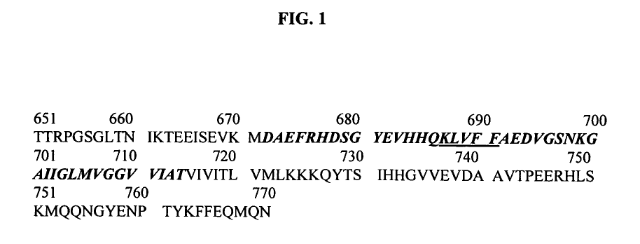

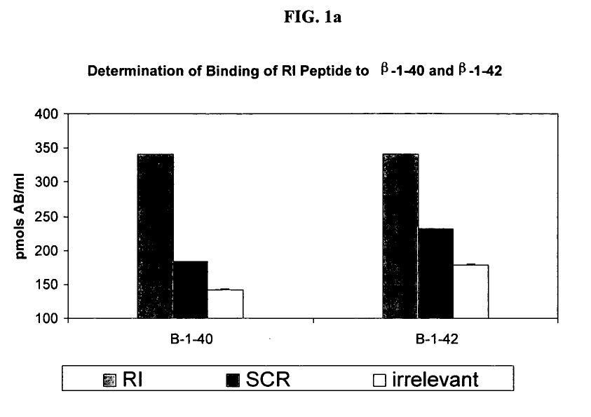

Testing the binding and stability of biotinylated Aβ peptides in binding solution. The ability of the biotinylated, nt-bAP, which is represented by Aβ peptides (1-42 and 1-40) to bind the binding element, the retro inverso peptide (RI) was investigated on a direct ELISA as described at the end of this section. RI, scrambled or an irrelevant peptide, immobilized on an ELISA plate was allowed to bind biotinylated Aβ peptides, 1-42 or 1-40. These results showed significant and specific binding to RI peptide when compared to the scrambled or an irrelevant control peptide (FIG. 1a). The results demonstrated that the 6-mer peptides that we designed and the biotinylated Aβ¹⁻⁴² and biotinylated Aβ¹⁻⁴⁰ that we purchased from a commercial vendor were authentic and functional since the peptide: peptide binding assay worked as we designed. Further, the biotinylation of Aβ¹⁻⁴² and Aβ¹⁻⁴⁰ did not interfere with their binding to RI peptide and this was in agreement with the product specifications from the vendor. These results validated the quantitation assay that we designed.

Next we investigated the effect of BSA and the stability of biotinylated Aβ peptide in binding solution over a period of time. Our binding assay measures the decrease in biotin levels in the surrounding liquid. This decrease could be due to reduction in the level of biotin caused either by breakdown of biotin from the biotinylated Aβ peptide or by binding onto the walls of the assay wells. Therefore, testing the stability in binding solution was necessary. {Initial experiments performed with Aβ peptide and buffer revealed that significant portion of the biotinylated Aβ peptide was lost in the absence or presence of an empty gel. This led us to understand that the peptide was binding to the walls of the wells. Therefore, the plate wells used for the assay needed to be pre-coated with a mixture of proteins in order to prevent background binding of Aβ peptide to the walls. A coating step was introduced and was followed for all subsequent binding assays. The results of this experiment performed on pre-coated wells showed that there was no background binding and that the biotinylated Aβ peptide was stable for a period ranging from 4 hours to 24 hours (data not shown). Still, this is a tricky assay. Besides the problem of sticking to surfaces, the biotinylated peptide is undergoing a competing reaction, aggregation, either at the binding site in the gel or elsewhere in the plastic tube or even inside an empty gel. Thus, at each time point, all the buffer (ca. 1 ml) surrounding each gel was removed and sonicated, an aliquot (ca. 50 μL) was taken for measurement and the remainder plus 50 μL was returned to the gel.

### EXAMPLE 3

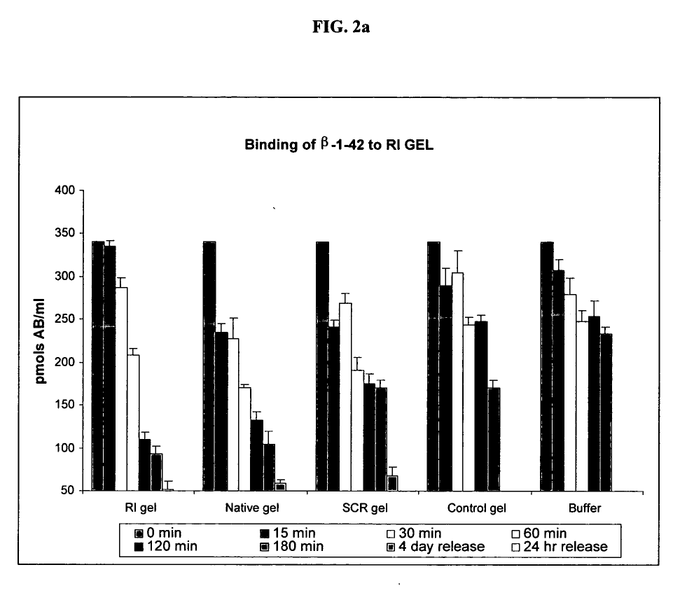

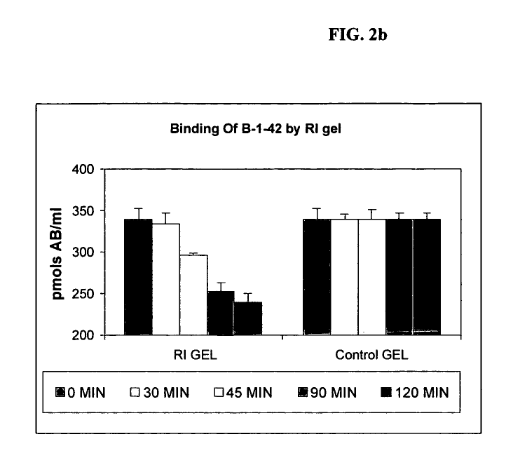

Binding of biotinylated Aβ¹⁻⁴² (less soluble) and Aβ¹⁻⁴⁰ (more soluble) peptides to detox gels was investigated. First a binding experiment for biotinylated Aβ¹⁻⁴² was performed with RI, Scrambled, native or control gel or no gel (buffer). Binding was allowed to continue for 3 hours while samples were harvested at designated time points. ELISA was performed to quantitate the levels of biotinylated Aβ¹⁻⁴² peptide left in the binding solution at the time of harvest. Results showed that the binding was steady and specific up and until 2 hour time point after which even the control gels appear to bind the peptide with a slower rate as compared with the RI gel (FIG. 2a). RI and native gels behaved in a similar fashion, as expected. Further, very low or no release of Aβ¹⁻⁴² peptide back into the medium could be detected even after 4 days. From these results it was inferred that the subsequent experiments be performed for a period of two hours and with only RI (detox) gel and an empty control gel. As a repeat test, a binding experiment was performed with RI and control gels and the results showed reproducible binding of biotinylated Aβ¹⁻⁴² peptide to detox but not control gel (FIG. 2b). In some experiments, the control gel, rather than being an empty gel, was a previously used gel that had been saturated with biotinylated Aβ¹⁻⁴².

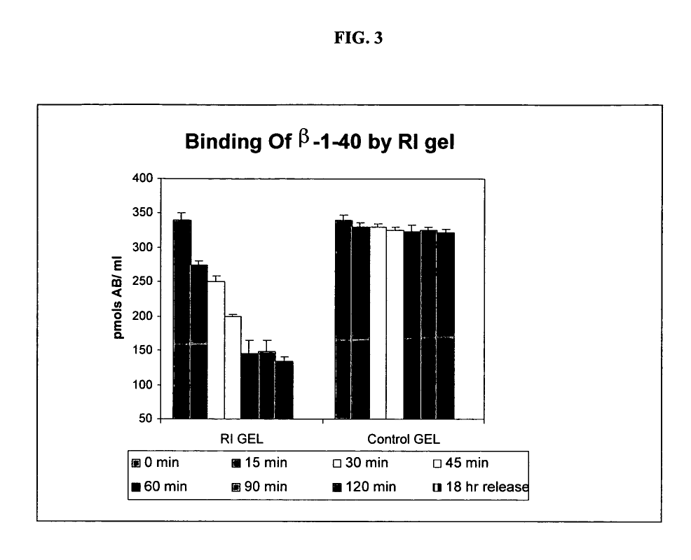

The binding experiment was then repeated under the same conditions except that Aβ¹⁻⁴² was replaced with Aβ¹⁻⁴⁰. The results showed that the detox (RI) but not the control gel bound Aβ¹⁻⁴⁰ efficiently. Similar to Aβ¹⁻⁴², the binding of Aβ¹⁻⁴⁰ to detox gel appeared irreversible since no release of Aβ¹⁻⁴⁰ could be detected after 18 h (FIG. 3).

### EXAMPLE 4

Determination of Aβ release. Simultaneously, the hydro gels were studied for the reverse process, leakage of Aβ back into the medium. At the end of each binding experiment, gels were washed twice in PB and placed in PB for many days. Samples of the media were harvested after every day and assayed for the presence of biotinylated Aβ peptide by ELISA as described above. The results showed that there was no or very little release after 18-24 hours of incubation in plain medium. Note that in many cases, there is no binding and the bar is not present in that graph since it has a value of zero. When a small amount of release occurs, the release data are represented in the chart for that experiment.

### EXAMPLE 5

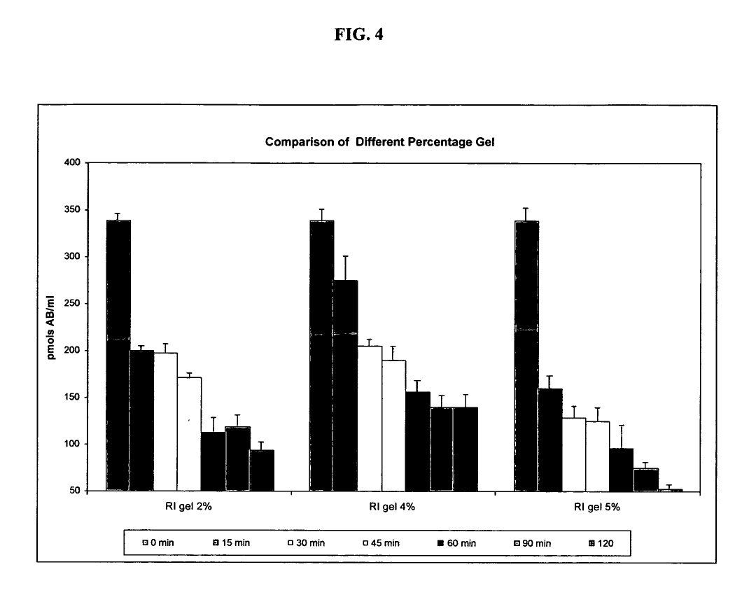

We performed a binding experiment using different percentage gels to see if the porosity of the gels makes any difference in the amount or the rate of binding of biotinylated Aβ¹⁻⁴² peptide. Binding of biotinylated Aβ¹⁻⁴² peptide to 2%, 4% and 5% detox gels was examined. The results showed that within the range of concentrations tried, porosity did not significantly influence or interfere with the binding property (FIG. 4).

### EXAMPLE 6

Amino Acid Analysis. We performed amino acid analysis (AAA) as a way of evaluating the RI: Aβ¹⁻⁴² binding directly. AAA would confirm the presence of Aβ¹⁻⁴² peptide in detox gels after the binding experiment. Therefore, representative gels (empty and RI gels, pre- and post-binding) were washed in HPLC grade water and sent over to WB Keck Foundation for Biotechnology at Yale University. There were large background signals from the PEG gels. The background from empty gel was used to normalize the results from RI gels pre- and post-binding experiment. At the Keck laboratory the gels were digested in 6 N HCl. Any peptide present in the gel would be hydrolyzed into its constituent amino acid subunits, which are then analyzed, by ion-exchange chromatography and post-column reaction with ninhydrin. In our application, this method is being pushed to its limit of detection and its accuracy due to false peaks generated from the gel background. Still, after subtracting data from a blank gel we can deduce the following.

A gel containing RI peptide gave the results: valine (10 nmols), leucine (9.6 nmols) and phenylalanine (19 nmols). These values (1.0:0.96:1.9) agree with the molar ratios (1:1:2) in the RI peptide, phe-phe-val-leu-lys. Lysine could not be measured due to high background, but the hydrophobic amino acids elute in a clear region of the chromatogram. We can also deduce that the absolute amount of RI peptide in the gel is 9.8 nmols (average of the 3 amino acids).

A gel containing RI peptide and incubated in biotin-Aβ¹⁻⁴² gave similar results, except there was, in addition, about one-tenth the amount of the hydrophobic amino acids, isoleucine and tyrosine, which are in the Aβ¹⁻⁴² peptide but not in RI peptide. We deduce that the gel had captured between 2% (based on isoleucine) and 15% (based on tyrosine) of the Aβ¹⁻⁴² peptide, which is between 200 and 1,400 pmols. The value according to ELISA was typically 200 to 250 pmols. In conclusion, this can be a valuable analytical tool to provide direct evidence for the RI: Aβ binding, but it requires further development and validation.

The goal was accomplished. It was possible to make a detoxification depot. Further proof from in vivo studies will be forthcoming.

What is claimed is:

1. A composition of matter comprising a biocompatible matrix in the form a of hydrogel through which water and other substances can diffuse and a matrix-linked capture reagent for the neurotoxic beta-amyloid peptides (nt-bAP) associated with Alzheimer's disease.

2. The composition of matter defined by claim 1, wherein the hydrogel and the capture reagent comprise a depot which is administered one of subcutaneously and intradermally to a patient with Alzheimer's disease.

3. The composition of matter defined by claim 1, wherein the depot comprises polyethylene glycol polymer chains that are cross-linked.

4. The composition of matter defined by claim 1, wherein the depot forms in situ after injection of a solution.

5. The composition of matter defined by claim 1, wherein the ability to extract beta-amyloid peptides is due to the presence of a monoclonal antibody, single chain antibody, fragment of an antibody or other derivative of an antibody.

6. The composition of matter defined by claim 1, wherein the ability to extract neurotoxic beta-amyloid peptides (nt-bAP) is due to the presence of KLVFF-related peptides covalently linked to the matrix of the depot.

7. The composition of matter defined by claim 6, wherein the KLVFF-related peptide is the retro-inverso analog composed of D-amino acids in the reverse sequence, ffvlk.

8. The composition of matter defined by claim 6, wherein the KLVFF-related peptide is linked to the matrix through its N-terminus.

9. The composition of matter defined by claim 6, wherein the KLVFF-related peptide is linked to the matrix through its C-terminus.

10. The composition of matter defined by claim 6, wherein the KLVFF-related peptide is linked to the matrix through a linker molecule.

11. The composition of matter defined by claim 6, wherein the KLVFF-related peptide is linked to the matrix.

12. The composition of matter defined by claim 6, wherein substitutions, additions, deletions or other modifications are present in the KLVFF-related peptide, either in the backbone or the side chains or in both, that do not materially alter the beta-amyloid binding properties.

13. The composition of matter defined by claim 6, wherein the KLVFF-related peptide is linked to a polymer molecule that is physically trapped in the depot matrix rather than covalently linked to the depot matrix.

14. The composition of matter defined by claim 6, wherein the KLVFF-related peptide is a repeating dimer, trimer or higher multimer that is then appended at one position to the depot matrix.

15. The composition of matter defined by claim 6, wherein monomer, dimmer, trimer or other multimers of KLVFF-related peptides can interact with one another to bind to nt-bAP.

16. The composition of matter defined by claim 1, wherein one of a protease and peptidase is incorporated into the depot.

17. The composition of matter defined by claim 1, wherein the depot comprises bioreversible bonds that allow the depot to autodegrade.
